(19) United States
(12) Patent Application Publication
(10) Pub. No.: US 2005/0058685 A1
(43) Pub. Date: Mar. 17, 2005

(54) Method of immobilization of clusters of ligands on polymer surface and use in cell engineering

(76) Inventors: Hai-Quan Mao, Baltimore, MD (US); Chao Yin, Wuhan (CN); Ron-Xi Zhuo, Wuhan (CN); Kam W. Leong, Ellicott City, MD (US)

Correspondence Address: Claude Nassif;Greenberg Traurig, Suite400E, 2450 Colorado Avenue, Santa Monica, CA, 90404, US

(21) Appl. No.: 10/494,830

(22) PCT Filed: Sep. 6, 2003
(86) PCT No.: PCT/SG03/00212

Related U.S. Application Data
(60) Provisional application No. 60/408,789, filed on Sep. 6, 2002.

(51) Int. Cl. A61F 2/00; C12N 5/02

(52) U.S. Cl. 424/426; 435/382

(57) ABSTRACT

The present invention describes a method to immobilize high density of cell-specific ligands on polymeric surface for cell engineering applications. This method combines a surface-grafting polymerization procedure to yield high density of functional groups, and a chemical conjugation step to link cell-specific ligands to the surface functional groups. This surface functionalization scheme can be applied to polymeric materials in various forms, such as polymer membrane, film, fiber, hollow fiber, foam, etc. Tissue-engineering scaffolds can be functionalized with cell specific ligand in the same manner.

### RELATED APPLICATIONS

This application is related and claims priority to U.S. Provisional Application Ser. No. 60/408,789, filed Sep. 6, 2002 and entitled “Method of Immobilization of Clusters of Ligands on Polymer Surface and Use in Cell Engineering”, herein incorporated by reference in its entirety.

### FIELD OF INVENTION

The present invention generally relates to functionalization of polymeric materials for cell and tissue engineering applications. It involves a method of immobilizing high density of cell-specific ligands to polymeric materials surfaces. These surface-functionalized polymeric materials (2-D or 3-D; non-biodegradable or biodegradable) are designed for engineering cells or tissues.

### BACKGROUND AND PRIOR ARTS

Acute liver failure (ALF) is a life-threatening disease with mortality as high as 70% and claims over 30,000 deaths per year in the United States. Liver transplantation may be the best course for most ALF patients, but acute shortage of donors prevents the widespread application of this approach¹. Under intensive care to prevent or manage life-threatening nonhepatic complications, a fraction of the patients with residual liver function still capable to sustain life may recover. There is another group of patients whose livers are not capable of providing the vital functions but still can regenerate if the patients are kept alive by temporary liver support systems. A bioartificial liver assist device (BLAD) aims to provide such a support system to temporarily substitute liver functions and sustain the patient's life until donors become available or the failed liver regenerates. A BLAD consists of three key components: cells which exhibit the important hepatic phenotype, a scaffold/substrate which promote and prolong the expression of these phenotypes, and a bioreactor which facilitates the biochemical reactions and biological transport between the hepatocytes and the perfused blood^{2,3}.

One of the determinant factors for the successful BLAD design is a suitable scaffold or substrate for hepatocyte attachment and functional maintenance, as hepatocytes are anchorage-dependent cells⁴. Various extracellular matrix (ECM) proteins, such as collagen^{5,6}, fibronectin⁷ and laminin⁸ or cell adhesion peptides, such as RGD and YIGSR⁹ have been used as substrates for hepatocyte culture. The attachment and viability of hepatocytes can be improved by culturing on surfaces coated with these ECM proteins. It is believed that this improvement is due to the specific interaction between cell adhesion peptides (e.g. RGD) in ECM proteins and the integrin receptors on hepatocyte surface, although the mechanism remains unclear. Changing the culturing configuration to three-dimensional by using collagen gel or Matrigel™ overlay could significantly improve the maintenance of hepatic function and longevity of primary rat hepatocytes¹⁰. Nevertheless, applying such a hydrogel system into a bioreactor design is a great challenge.

Galactosylated surface is an attractive alternative as a hepatocyte culture substrate because of the specific interaction between the galactose ligand and asialoglycoprotein receptor (ASGP-R) on hepatocyte surface. Several studies have shown that immobilized galactose ligand could improve hepatocyte attachment and sustain their cellular functions¹¹⁻¹³. This includes polyacrylamide gel immobilized with galactopyranosyl group¹¹ and polystyrene surface coated with poly-N-vinylbenzyl-D-lactonamide (PVLA)¹⁴. The latter surface has been shown to increase the hepatocyte adhesion and maintain the albumin synthesis function for 2 weeks¹⁴. This effect was dependent upon the surface density of galactosyl ligand (or PVLA). Surface coated with low density of PLVA (corresponding to 0.15 nmol/cm² galactose) yields spread morphology and low level of bile acid secretion, whereas higher density of PVLA coating (corresponding to 2.3 mmol/cm² galactose) maintains round morphology and induces the formation of hepatocyte aggregates, which expresses high level of bile acid secretion and long-term viability^{15,16}. It is a challenge to create a substrate with high galactose ligand density that can facilitate high affinity adhesion and high-density cell culture for optimal BLAD performance.

In this study, we developed a surface modification strategy to achieve a high density of immobilized galactose ligand using a polyacrylic acid (PAA) grafting technique (FIG. 1). The galactose conjugation and the binding activity of surface immobilized galactose ligand were characterized. Effects of surface galactose moiety on hepatocyte attachment, morphology and functions were investigated using a high-density hepatocyte culture in comparison with collagen-coated substrate.

### SUMMARY OF THE INVENTION (PREFERRED EMBODIMENTS)

This invention describes two methods for immobilization of high density of cell specific ligands to polymer surfaces:

The first immobilization method includes the following three steps:

(1). Polymeric materials in the forms of 2-dimenional or 3-dimensional structure (e.g. films, sheets, membranes, fibers, hollow fibers, forms, woven, knitted, braided fibers, etc.) is first activated by plasma treatment (under argon, oxygen, amonia, etc.) or ozone treatment or UV radiation etc. to generate free radials or functional groups on the surface.

(2). These pre-activated surfaces are reacted with a monomer solution to graft polymer chains with functional groups, such as carboxyl groups, amino groups, hydroxyl groups, sulfhydryl groups, etc. The free radials or functional groups on the surface that are generated during the first step serve as initiators for the grafting polymerization.

(3). A ligand for specific cell types is conjugated to the functional groups generated by the second step via a chemical linkage. Different chemistries can be used for the ligand conjugation depending on the functional groups on the ligand and the polymer surface.

The second method includes two steps:

(1). A polymer chain (with functional groups, e.g. carboxyl groups and amino groups) is grafted to the polymer surface under UV initiation directly. For example, a PET membrane is submerged in an acrylic acid solution (0.01% to 10%) and is subjected to UV irradiation for 5 to 10 minutes, followed by extensive washing with water.

(2). A hepatocyte specific ligand is conjugated to the functional groups generated by the second step via a chemical linkage. Different chemistries can be used for the ligand conjugation depending on the functional groups on the ligand and the polymer surface. Typically an EDC coupling reaction is used to conjugate the ligand to the functionalized surface.

Applications of the hepatocyte specific biofunctional substrates and scaffolds involve culturing hepatocytes (either primary hepatocytes or hepatic cell lines) in the scaffolds/substrates in an optimized medium (serum free or serum containing medium).

As the following examples show, using polyacrylic acid (PAA) grafting technique coupling with the galactose ligand conjugation scheme, we have successfully developed a hepatocyte-specific surface with high surface-ligand density and high ligand mobility. Such a galactosylated PET substrate supported efficient cell attachment and stimulated hepatocyte aggregate formation. Albumin synthesis function of the hepatocytes cultured on this biofunctional substrate was better maintained compared with that on collagen-coated surface. This study suggested the potential application of this hepatocyte-specific biofunctional substrate in liver tissue engineering, particularly for the design of bioartificial liver assist device.

### DESIRABLE FEATURES OF PROPOSED INVENTION

Extracellular matrix is known to play a key role in the phenotypic maintenance of hepatocytes^{17,18}. Collagen coated¹⁹ and galactose-containing polymer (PVLA) coated surfaces¹⁵ yielded dramatically different cellular behavior. Hepatocytes cultured on collagen coating formed a fattened monolayer, whereas PVLA coating promoted rounded morphology and aggregate formation. These studies were performed using a relatively low cell seeding densities, presumably due to the relatively low surface ligand concentration. This report provided a surface modification strategy leading to a much higher amount of ligands on the substrate surface. The scheme involved surface plasma treatment to grafting PAA chains to PET film surface, followed by conjugation of galactose ligand.

Graft polymerization is an effective approach to modify materials surface²⁰. It has been used widely to functionalize surfaces to improve biocompatibility of materials. Graft polymerization procedure also allows the flexibility of adjusting the extent of modification. In this study, conditions were optimized to introduce a high amount of carboxyl groups on the surface through PAA grafting. This in turn resulted in a high surface density of the galactose ligand (0.513 μmol/cm²). The highest ligand density is about 220 times higher than that achieved by PVLA coating on polystyrene surface (galactose density of 2.3 nmol/cm²).

In this design, the PAA chain also serves as spacers to provide high mobility for the conjugated ligands. This feature, combined with the high ligand density, led to high bind affinity of the surface conjugated ligands, as demonstrated by the FITC-lectin binding experiment. Lectins, including the rat asialoglycoprotein receptor, are proteins that specifically bind carbohydrate ligands, comprised of three types of subunits and multiple binding sites. Studies have revealed that the binding affinity between carbohydrate and lectins can be increased by several orders of magnitude through the clustered multiple binding, comparing with the monovalent mode^{21,22}. We hypothesized that the high concentration of galactose and high nobility of the surface galactose ligands resulted in many such ligand-clusters on the surface, therefore could mediate high attachment for hepatocyte²³. This hypothesis is supported by the results showing that binding between the galactosylated PET film and the FITC-lectin could not be inhibited significantly by excess amount (150 folds) of free galactose in the solution.

Hepatocyte aggregation formation and functional maintenance are ligand density dependent (FIG. 6). Hepatocyte attachment efficiency on PET-Gal surface increased with the surface ligand density, and reached a plateau of 78% (Gal density>47 nmol/cm²), which was the same as that on collagen coated surface. Extensive aggregation formation was obvious when the ligand density exceeded 9 nmol/cm²; whereas the urea synthesis function reached the plateau at a surface ligand density of around 48 nmol/cm². In concomitance to the spheroid formation, urea synthesis function of hepatocytes attached to substrates with higher ligand density (47.5-513 nmol/cm²) was higher than that attached on to surfaces with lower ligand density.

This ligand immobilization method can be widely used for many cell and tissue engineering applications to conjugate cell specific ligand at desired density. The generality of this approach implies the wide applicability of this scheme to various polymeric materials, both biodegradable and non-biodegradable polymers, either in 2-dimensional or 3 dimensional configurations. An example is of another material is poly(ε-carprolactone-co-ethylene ethyl phosphate) [P(CL-co-EEP)], a biodegradable polymer. Similar to the galactosylated PET substrate, galactosylated P(CL-co-EEP) substrate showed a much higher cell attachment rate (90.2%) than the unmodified substrate (50.4%). The collagen coated P(CL-co-EEP) membrane showed a cell attachment rate of 81.7%. Hepatocytes cultured on the galactosylated P(CL-co-EEP) substrate formed spheroids, in contrast to die sparking adhesion on day 3 on the unmodified surface (FIG. 7). Collagen coated membrane stimulated the monolayer formation, as expected.

Cell-substrate interaction is a key parameter in regulating cellular behavior on many tissue-engineering systems. Understanding the mechanism of the regulation of hepatocyte behavior by substrate is crucial to the design of a suitable scaffold to maintain optimal hepatocyte functions. A chemically well-defined substrate described in this report would provide an excellent model for mechanistic study. It would allow systematic evaluation of various factors involved in this cell-substrate interaction, for example, type of ligand, ligand density and multi-ligand synergy.

### BRIEF DESCRIPTION OF THE DRAWINGS

FIG. 1. Surface modification scheme for galactose conjugation to PET film.

FIG. 2. Galactose ligand conjugating efficiencies achieved using different EDC addition mode: (A) 50 mg/mL EDC added before reaction; (B) 30 mg/mL EDC added before reaction; (C) 10 mg/mL EDC added daily for 3 days; (D) 20 mg/mL EDC added daily for 3 days.

FIG. 3. Hepatocyte attachment on different surfaces: TCPS: Tissue culture polystyrene surface; PET: unmodified PET film; PET-COOH: PAA-grafted PET film; PET-Gal: galactose-conjugated PET film, TCS-Collagen: Collagen-coated TCPS surface.

FIG. 4. Morphology of hepatocytes cultured on different substrates at various time points (legend see FIG. 3).

FIG. 5. Albumin synthesis function of hepatocytes cultured on different substrates as a function of culture time (legend see FIG. 3).

FIG. 6. Hepatocyte aggregation formation and functional maintenance are ligand density dependent

FIG. 7. Formation of spheroids in on poly(ε-carprolactone-co-ethylene ethyl phosphate.

### EXAMPLES

The following examples are offered by way of illustration and are not intended to limit the invention in any manner.

### Example 1

### Immobilization of High Density of Galactose Ligand on Polyethyleneterephthalate (PET) Membrane Surface (FIG. 1)

PET films with a thickness of 100 μm were purchased from Goodfellow (UK). Acrylic acid (AAc) and galactose was purchased from Merck (Germany). N-hydroxysuccinimide (Sulfo-NHS) was purchased from Pierce (USA). All other chemicals were purchased from Sigma-Aldrich unless otherwise stated.

PET Surface Grafting With Polyacrylic Acid (PAAc)

The PET film was cut into pieces with a dimension of 2.5 cm×5 cm, and cleaned with alcohol for 5 min in an ultrasonic water bath. These PET films were placed between the two parallel plate electrodes of a quartz cylindrical-type glow discharge cell (Model SP100, Anatech Ltd., USA) and subjected to the glow discharge for 30 sec under an argon pressure of 0.5 Torr. The plasma power and radio frequency were kept at 30 W and 40 kHz, respectively. The Ar plasma-treated PET films were then exposed to oxygen gas for 30 min, and immersed in 30 mL of an aqueous solution containing 10 vol. % of acrylic acid (AAc) in Pyrex® tubes. The AAc solution was thoroughly degassed using Ar for 30 min and sealed under Ar atmosphere. The reaction mixture was subjected to UV irradiation for 30 min using a 1000 W high-pressure mercury lamp in a rotary photochemical reactor (Riko RH400-10W, Riko Denki Kogyo of Chiba, Japan). A water bath was used to keep the reaction temperature at 25° C. After graft polymerization, the PET films were removed from the tubes and washed extensively with water in a Soxhlet extractor.

Synthesis of 1-O-(6′-Aminohexyl)-D-Galactopyranoside (AHG)

The galactose ligand AHG was synthesized according procedures reported in the litertures^{24,25} with slight modification.

Conjugation of AHG to PAA-Grafted PET Films

PAA-grafted PET films were cut into round discs with a diameter of 15 mm. Each disc was immersed in sodium phosphate buffer (0.1 M, pH 7.0, 1 mL) with 2 mg of AHG and 1 mg of Sulfo-NHS. Different amount of 1-ethyl-3-(3-dimethylaminopropyl)-carbodiimide hydrochloride (EDC) was added to each well and the mixture was reacted for 3 days. To optimize the coupling efficiency, different EDC addition modes were tested: (A) 50 mg/mL at the beginning of reaction; (B) 30 mg/mL at the beginning of reaction; (C) 10 mg/mL daily for three days; (D) 20 mg/mL daily for three days.

Determination of Carboxyl Group Density.

Samples of modified PET films with surface area of 3 mm² were incubated with 1 mL toluidine blue O(TBO) solution (0.5 mM) in 0.1 mM NaOH (pH10) under constant shaking for 5 h at room temperature. Un-complexed dye was removed by washing with excess amount of 0.1 mM NaOH solution. The complexed TBO on PET films was desorbed from the surface by incubating the sample in 1 mL of 50% acetic acid solution for 10 min under vortexing. TBO concentration in acetic acid solution was determined by its optical density at 633 nm with a Beckman spectrophotometer DU640B. Carboxyl group density on the surface was calculated from the complexed TBO content assuming that TBO complexes with carboxyl acid at 1:1 ratio²⁶.

X-Ray Photoelectron Spectroscopy (XPS).

XPS measurements were made on a Kratos AXIS HSi spectrometer with a monochromatized Al KR X-ray source (1486.6 eV photons) at a constant dwelling time of 100 ms and a pass energy of 40 eV. The anode current was 15 mA The pressure in the analysis chamber was maintained at 5.0×10⁻⁸ Torr for each measurement.

Results:

The PET films were treated by low-temperature plasma at first and subsequently exposed in oxygen to introduce peroxide groups to the surface of the PET membrane. Under UV light, peroxide groups were activated to form radical-initiating center, which then initiated the graft polymerization of AAc. The degree of PAA grafting was modulated by power and duration of plasma treatment, intensity and duration of UV treatment, and die concentration of acrylic acid solution. Under the optimized condition, we achieved an average carboxyl group density of 530 nmol/cm² (38.7 μg/cm²) on the PAA-grafted PET films as determined by Toluidine Blue O staining.

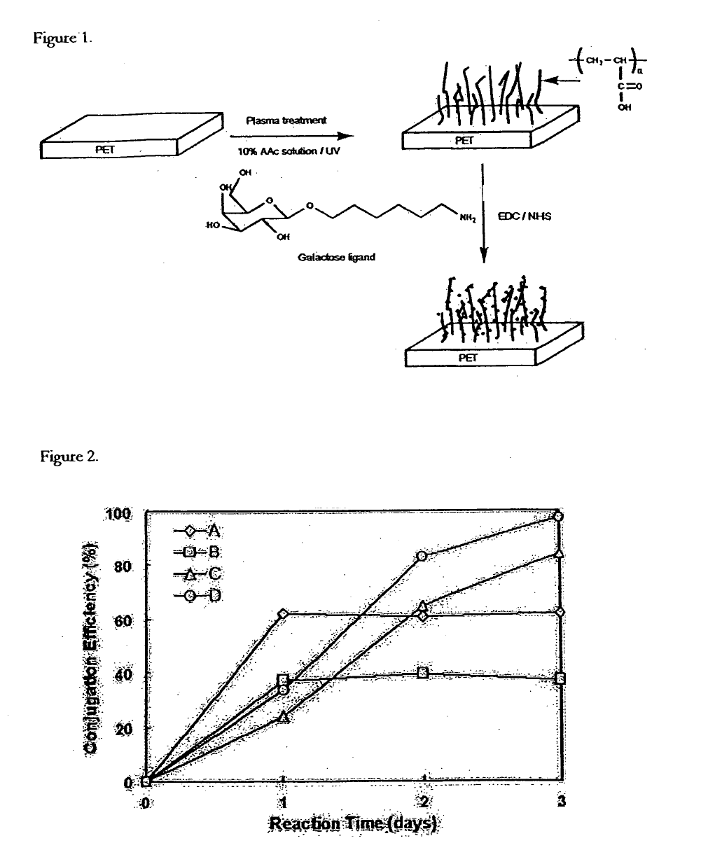

To conjugate a galactose moiety to the carboxyl groups on PET surface, we have used a galactose derivative with aminohexyl group linked to the C¹ position of the galactose ring. This AHG derivative afforded a short spacer between the galactosyl group and the conjugating point on PAA chain to facilitate the conjugation reaction and increase the accessibility of the ligand to cell receptors. The terminal amino group in AHG allowed its conjugation to surface carboxyl-group through standard EDC chemistry²⁷. This condensation reaction was carried out with the aid of Sulfo-NHS. EDC loses its activity-very quickly in the aqueous solutions. Sulfo-NHS was added to increase the stability of the active ester intermediate and ultimately improve the conjugation efficiency. The conjugation efficiency was determined by analyzing the surface carboxyl content change before and after the reaction. To optimize the coupling condition and achieve the highest conjugation efficiency, EDC was added in the reaction solution in different modes—adding in three portions over three days versus in one feed at the beginning of the reaction. The result of AHG conjugation efficiency was shown in FIG. 2. The concentration and addition mode of AHG significantly affected its conjugation efficiency. At an EDC concentration of 50 mg/mL, a 62% conversion of surface carboxyl groups was achieved after reacting for one day, while only 24% conversion was obtained at an EDC concentration of 10 mg/mL. Additional reaction time beyond one day did not improve the conversion rate of die surface carboxyl group. The highest coupling efficiency (97%) was achieved when 20 mg EDC was added to die reaction solution (1 mL) daily for 3 days. Carboxyl content on the galactosylated PET film was only 17 nmol/cm² under this condition. This corresponded to 513 nmol/cm² of galactose ligand on the surface.

### Example 2

### Binding Affinity of Surface Conjugated Galactosyl Group to FITC-Lectin

Samples of PET films with surface area of 3 mm² were incubated with 250 μL of FITC-lectin solution (from psophocarpus tetragonolobus, 250 μg/mL) for 1 h at 37° C. The samples were washed extensively with water, and observed under an Olympus FLUOVIEW confocal microscope (εₑₓ 488 nm). In a parallel experiment, free galactose was added to the FITC-lectin solution to yield a final concentration of 5 mg/mL before incubating with the modified PET film.

FITC-lectin was used to confirm the binding activity of the surface conjugated galactose ligands. The lectin used in this study was from psophocarpus tetragonolobus and could specifically bind galactose or N-acetyl-D-galactosamine. FITC labeled lectin was used in order to visualize the binding under confocal fluorescence microscope. Different PET films (unmodified, PAA-grafted and galactosylated) were incubated with FITC-lectin for one hour at 37° C. and washed extensively before viewing. Confocal fluorescence images of these films showed that the galactosylated PET film showed high FITC fluorescence, whereas the unmodified PET film and PAA-grafted PET film yielded no noticeable fluorescence.

A competition experiment was performed using free galactose added to the lectin solution in order to estimate the binding affinity between surface conjugated galactose ligands and FITC-lectin. Galactosylated PET film was incubated with FITC-lectin together with 5 mg/mL of free galactose, corresponding to 150 folds higher amount of free galactose ligand than that on the modified PET film. Fluorescence image of the galactosylated PET film incubated with FITC-lectin in the presence of free galactose indicated no decrease in fluorescence intensity when comparing with that of galactosylated PET film incubated with FITC-lectin in the absence of free galactose. This competitive binding could not be the result of adsorption of either FITC-lectin or the complex of FITG-lectin/galactose, since the parallel tests with unmodified and PAA-grafted PET membranes incubating with FITC-lectin yielded negligible fluorescence on the membrane. This result suggested that the surface conjugated galactose ligands had a much higher binding affinity than soluble galactose to its receptor.

### Example 3

### Attachment and Morphology of Hepatocytes Cultured on Different Surfaces

Hepatocytes were harvested from male Wister rats weighting from 250 to 300 g by a two-step in situ collagenase perfusion as described previously²⁹. NIH guidelines for the care and use of laboratory animals (NIH Publication # 85-23 Rev. 1985) have been observed. Hepatocyte viability was determined to be 90-95% using the Trypan Blue exclusion method.

Unmodified and modified PET films were cut into round discs with a diameter of 15 mm and fixed to the wells of a 24-well tissue culture plate with 5-10 μL of chloroform. The plate was sterilized by incubating with 70% ethanol for 3 h and washed with PBS for 3 times. Collagen-coated surface was used as a control and was prepared by placing 0.5 mL of collagen solution (0.5 mg/mL in PBS) in each well, leaving the plate overnight at 4° C. The solution in each well was aspirated and the wells were washed with PBS for 3 times.

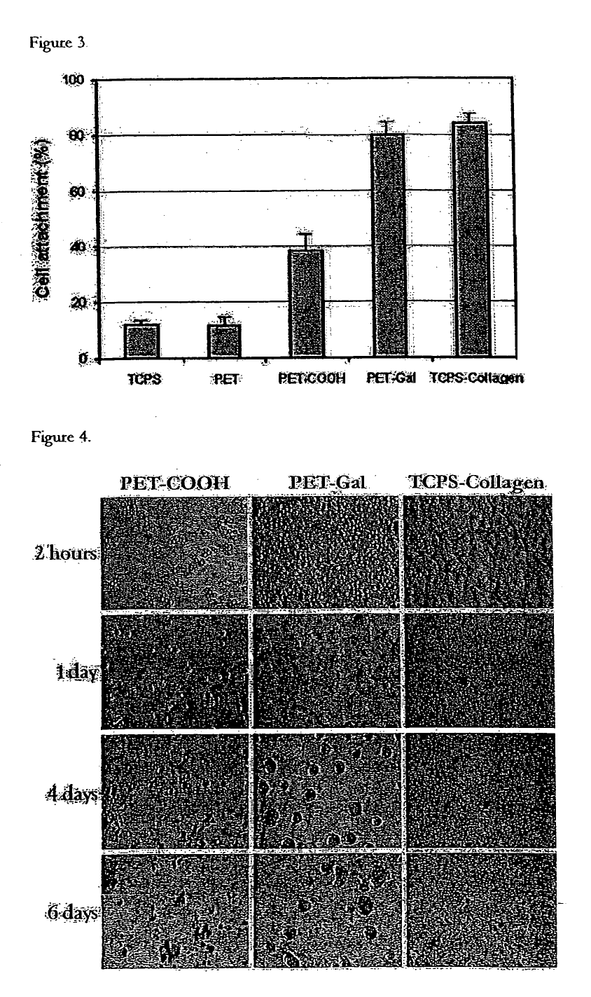

Five types of surfaces were used in the cell attachment determination: tissue culture polystyrene (TCPS), collagen-coated TCPS, unmodified PET, PAA-grafted PET and galactose-immobilized PET. Freshly isolated hepatocytes were suspended at a density of 1.2×10⁶ cells/mL in William's Eagle medium supplemented with 1 mg/mL BSA, 100 units/mL penicillin and 100 μg/mL streptomycin, and dispensed on the different surfaces at 300 μL per well. This yielded a cell culture density on the surfaces of 2×10⁵ cells/cm². Cells were cultured in a humidified incubator with 5% CO₂. After 1 hour, the medium containing unattached cells was aspirated. The number of unattached hepatocytes was determined by a hemocytometer. Cell attachment rate was calculated from the number of attached hepatocytes on the surfaces and the seeding density. The results were shown in FIG. 3. After one hour of incubation at 37° C., hepatocytes exhibited low affinity to TCPS and unmodified PET surface with an attachment rate of below 15%. PAA-grafted PET surface showed a higher cell attachment (39%) than TCPS and PET membranes. In contrast, cell attachment on galactosylated PET surface was twice as that on PAA conjugated PET surface (80%), approaching that on collagen-coated TCPS (84%).

The attached hepatocytes on different surfaces were cultured for 6 days in William's E medium supplemented with 1 mg/mL BSA, 10 ng/mL of EGF, 0.5 μg/mL of insulin, 5 nM dexamethasone, 50 ng/mL linoleic acid, 100 units/mL penicillin and 100 μg/mL streptomycin. The medium was replenished daily. The collected medium was centrifuged in 14,000 rpm for 10 min and the supernatant was stored at −20° C. for albumin assay. At various time points of culture, morphology of hepatocytes on these substrates was viewed under an inverted microscope (Carl Zeiss) with phase contrast optics and recorded on a digit camera.

Hepatocytes cultured on different surfaces exhibited different morphology (FIG. 4). Two hours after cell seeding, hepatocytes on PAA-grafted and galactosylated PET films maintained the round morphology, whereas hepatocytes attached on collagen-coated TCPS began to spread out at this time. After one-day culture, hepatocytes on PAA-grafted and galactosylated PET films adopted different morphologies. On galactose-conjugated PET films, hepatocytes formed aggregates, but hepatocytes on PAA-grafted PET films remained relatively unchanged. At this time, cells on collagen-coated TCPS formed a confluent monolayer. The difference in morphology of the hepatocytes on different surfaces became more obvious in days 3-7. Hepatocytes on PAA-grafted PET films appeared mostly as rounded single cell with a few small aggregates of several cells at most. On the contrary, hepatocytes on galactosylated surface had the highest ability to migrate, forming large aggregates. Very few isolated cells or small aggregates remained on the surface. On collagen-coated surface, hepatocytes maintained the characteristic monolayer morphology. Cells with double nuclei became apparent at day 5, indicating that the hepatocytes were undergoing the proliferation phase.

### Example 4

### Albumin Synthesis Function of Hepatocytes Cultured on Different Surfaces

The albumin concentration in the culture medium collected at various time points was determined with a competitive enzyme linked immunosorbent assay (ELISA) analysis according to a protocol reported by Friend J R et al³⁰. Briefly, samples were serially diluted, and peroxidase conjugated rabbit antibody against rat albumin (ICN, USA) was added to a final concentration of 0.6 μg/mL. After incubation at 37° C. for 2 h, 100 μL aliquots of each sample were transferred to 96-well Maxisorp plates (Nunc Inc., USA), which were precoated by incubating with 100 μl/well of rat albumin at 0.2 μg/mL in PBS overnight at 4° C., and washed three times with 0.05% Tween-20 in PBS before use. The samples were incubated in the wells at room temperature for 2 h in a humidified chamber. Subsequently the plates were washed three times with 0.05% Tween-20 in PBS and filled with 100 μl/well 1-Step Turbo TMB-substrate (Pierce, USA). Plates were incubated at room temperature in the humidified chamber for 30-45 min and reaction was stopped by adding 100 μl of 2N H₂SO₄. The optical density (OD) at 450 nm of the solution in each well was determined on a microplate reader (Model 550, Bio-rad Laboratories). Rat albumin concentrations of the samples were calculated from the ODs using rat albumin as standards.

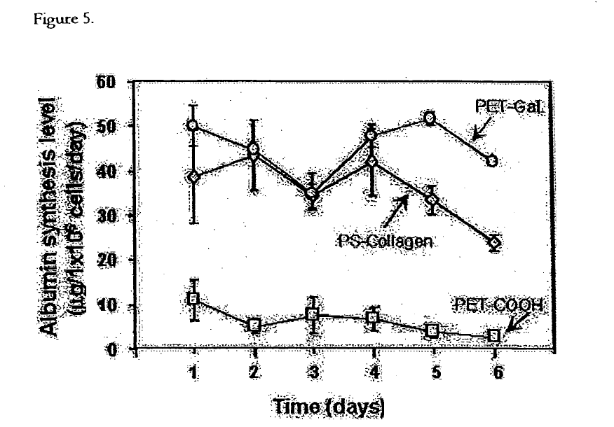

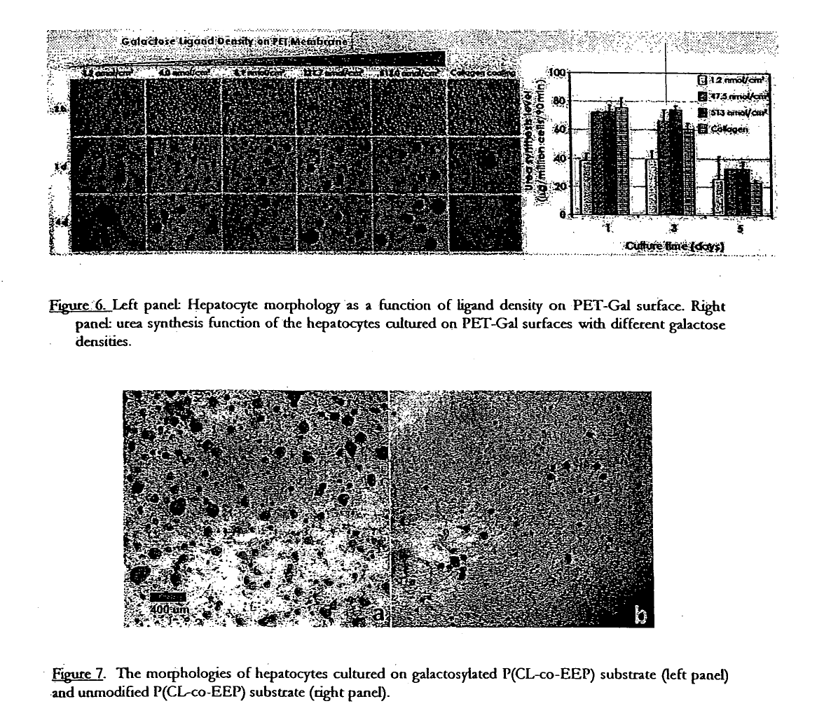

Hepatocyte function was evaluated by the albumin synthesis level as a function of time (FIG. 5). Albumin synthesis function of hepatocytes cultured on PAA-grafted substrate rapidly dropped to 10 μg/million cells/day on day 1 and continued to decrease over time. Hepatocytes cultured on the galactosylated PET surface and the collagen-coated surface exhibited similar levels of synthesis function for four days, followed by a gradual decrease on collagen-coated substrate. Synthesis function of hepatocytes on galactosylated PET surface was maintained at the same level for at least one week.

### REFERENCES

1. Tzanakakis E S, Hess D J, Sielaff T D, Hu W S. Extracorporeal tissue engineered liver-assist devices. Annu Rev Biomed Eng 2000; 2:607-632.
2. Allen J W, Hassanein T, Bhatia S N. Advances in bioartificial liver devices. Hepatology 2001; 34(3):447-455.
3. Sauer I M, Obermeyer N. Kardassis D, Theruvath T, Gerlach J C. Development of a hybrid liver support system. Ann N Y Acad Sci 2001; 944:308-19.
4. LeCluyse E L, Bullock P L, Parkinson A. Strategies for restoration and maintenance of normal hepatic structure and function in long-term cultures of rat hepatocytes. Adv Drug Deliv Rev 1996; 22:133-186.
5. Taguchi K, Matsushita M, Takahashi M, Uchino J. Development of a bioartificial liver with sandwiched-cultured hepatocytes between two collagen gel layers. Artif Organs 1996; 20(2):178-185.
6. Dunn J C, Yarmush M L, Koebe H G, Tompkins R G. Hepatocyte function and extracellular matrix geometry: long-term culture in a sandwich configuration. FASEB J 1989; 3(2):174-177.
7. Sanchez A, Alvarez A M, Pagan R, Roncero C, Vilaro S, Benito M, Fabregat I. Fibronectin regulates morphology, cell organization and gene expression of rat fetal hepatocytes in primary culture. J Hepatol 2000; 32(2):242-250.
8. Oda H, Nozawa K, Hitomi Y, Kakinuma A. Laminin-rich extracellular matrix maintains high level of hepatocyte nuclear factor 4 in rat hepatocyte culture. Biochem Biophys Res Commun 1995; 212(3):800-805.
9. Carlisle E S, Mariappan M R, Nelson K D, Thomes B E, Timmons R B, Constantinescu A, Eberhart R C, Bankey P E. Enhancing hepatocyte adhesion by pulsed plasma deposition and polyethylene glycol coupling. Tissue Eng 2000; 6(1):45-52.
10. Moghe P V, Berthiaume F, Ezzell R M, Toner M, Tompkins R G, Yarmush M L. Culture matrix configuration and composition in the maintenance of hepatocyte polarity and function. Biomaterials 1996; 17(3):373-385.
11. Weigel P H. Rat hepatocytes bind to synthetic galactoside surfaces via a patch of asialoglycoprotein receptors. J Cell Biol 1980; 87(3 Pt 1):855-861.
12. Kobayashi A, Kobayashi K, Akaike T. Control of adhesion and detachment of parenchymal liver cells using lactose-carrying polystyrene as substratum. J Biomater Sci Polym Ed 1992; 3(6):499-508.
13. Yang J, Goto M, Ise H. Cho C S, Akaike T. Galactosylated alginate as a scaffold for hepatocytes entrapment. Biomatetials 2002; 23(2):471-479.
14. Tobe S, Takei Y, Kobayashi K, Akaike T. Tissue reconstruction in primary cultured rat hepatocytes on asialoglycoprotein model polymer. Artif Organs 1992; 16(5):526-532.
15. Kobayashi A, Goto M, Sekine T, Masumoto A, Yamamoto N, Kobayashi K, Akaike T. Regulation of differentiation and proliferation of rat hepatocytes by lactose carrying polystyrene Artif Organs 1992; 16(6):564-567.
16. Kobayashi A, Goto M, Kobayashi K, Akaike T. Receptor-mediated regulation of differentiation and proliferation of hepatocytes by synthetic polymer model of asialoglycoprotein. J Biomater Sci Polym Ed 1994; 6(4):325-342
17. Berthiaume F, Moghe P V, Toner M, Yarmush M L. Effect of extracellular matrix topology on cell structure, function, and physiological responsiveness: hepatocytes cultured in a sandwich configuration. FASEB J 1996; 10(13):1471-1484.18. Iredale J P, Arthur M J. Hepatocyte-matrix interactions. Gut 1994; 35(6):729-732.
19. Mooney D, Hansen L, Vacanti J, Langer R, Farmer S, Ingber D. Switching from differentiation to growth in hepatocytes: control by extracellular matrix. J Cell Physiol 1992; 151(3):497-505.
20. Ikada Y. Surface modification of polymers for medical applications. Biomaterials 1994; 15(10):725-736.
21. Elgavish S, Shaanan B. Lectin-carbohydrate interactions: different folds, common recognition principles. Trends Biochem Sci 1997; 22(12):462-467.
22. Rice K G, Weisz O A, Barthel T, Lee R T, Lee Y C. Defined geometry of binding between triantennary glycopeptide and the asialoglycoprotein receptor of rat heptocytes. J Biol Chem 1990; 265(30):18429-18434.
23. Griffith L G, Lopina S. Microdistribution of substratum-bound ligands affects cell function: hepatocyte spreading on PEO-tethered galactose. Biomaterials 1998; 19(11-12):979-986.
24. Weigel P K, Naoi M, Roseman S, Lee Y C. Preparation of 6-aminohexyl D-aldopyranosides. Carbohydr Res 1979; 70:83-91.
25. Findeis M A. Stepwise Synthesis of A Galnac-Containing Cluster Glycoside Ligand of the Asialoglycoprotien Receptor. International Journal of Peptide and Protein Research 1994; 43(5):477-485.
26. Sano S, Kato K, Ikada Y. Introduction of functional groups onto the surface of polyethylene for protein immobilization. Biomaterials 1993; 14(11):817-822.
27. Hermanson G T. Bioconjugate Techniques. Academic Press, Inc., 1996. p 173-176
28. Nakajima N, Ikada Y. Mechanism of amide formation by carbodiimide for bioconjugation in aqueous media. Bioconjug Chem 1995; 6(1):123-130.
29. Chia S M, Leong K W, Li J, Xu X, Zeng K, Er P N, Gao S, Yu H. Hepatocyte encapsulation for enhanced cellular functions. Tissue Eng 2000; 6(5):481-495.
30. Friend J R, Wu I J, Hansen L K, Remmel R P, Hu W S. Formation and characterization of hepatocyte spheroids. In: Morgan J R, Yarmush M L, editors. Tissue engineering methods and protocols. Humana Press Inc; 1999. p 245-252.

1.) A substrate for cellular attachment and functional maintenance utilizing graft polymerization, comprising:
an at least two-dimensional polymeric material, said polymeric material having thereon at least one of free radicals or functional groups;
polymer chains grafted to said at least one of free radicals or functional groups; and a ligand conjugated to said at least one of free radicals or functional groups.

2.) The substrate for cellular attachment and functional maintenance of claim 1, wherein said at least two-dimensional polymeric material is selected from the group consisting of films, sheets, membranes, fibers, hollow fibers, forms, woven, knitted and braided fibers.

3.) The substrate for cellular attachment and functional maintenance of claim 1, said at least one of free radicals or functional groups is provided by activating a surface of said least two-dimensional polymeric material.

4.) The substrate for cellular attachment and functional maintenance of claim 3, wherein said activation is selected from the group consisting of plasma treatment, ozone treatment and UV radiation.

5.) The substrate for cellular attachment and functional maintenance of claim 4, wherein said plasma treatment is under at least one of argon, oxygen and ammonia.

6.) The substrate for cellular attachment and functional maintenance of claim 1, wherein said functional groups are selected from the group consisting of carboxyl groups, amino groups, hydroxyl groups and sulfhydryl groups.

7.) The substrate for cellular attachment and functional maintenance of claim 1, wherein said polymer chains are grafted to said polymer surface under UV initiation.

8.) The substrate for cellular attachment and functional maintenance of claim 1, wherein said polymer chains are comprised of polyacrylic acid.

9.) The substrate for cellular attachment and functional maintenance of claim 1, wherein said ligand is a galactose ligand.

10.) The substrate for cellular attachment and functional maintenance of claim 1, wherein said polymer chains serve as spacers providing high mobility for said conjugated ligand.

11.) The substrate for cellular attachment and functional maintenance of claim 1, wherein said polymer chains are comprised of poly(ε-carprolactone-co-ethylene ethyl phosphate).

12.) The substrate for cellular attachment and functional maintenance of claim 1, wherein said at least two-dimensional polymeric material is polyethylene terephthalate.

13.) A method for high density immobilization ligands to polymer surfaces, comprising:
providing a polymeric material in a 2 or 3-dimensional structure;
activating a surface of said polymeric material;
generating free radicals or a first set of functional groups on said surface;
initiating grafting polymerization to from polymer chains having a second set of functional groups, by reacting said free radicals or functional groups with a monomer solution; and
conjugating a ligand to said second set of functional groups.

14.) The method for high density immobilization ligands to polymer surfaces of claim 13, wherein said a polymeric material has at least one of a film, sheet, membrane, fiber and hollow fiber structure.

15.) The method for high density immobilization ligands to polymer surfaces of claim 14, wherein said fiber or hollow fiber is woven, knitted or braided.

16.) The method for high density immobilization ligands to polymer surfaces of claim 13, wherein said activating step of said surface of said polymeric material comprises plasma treatment under at least one of argon, oxygen and ammonia.
